(19) United States
(12) Patent Application Publication
(10) Pub. No.: US 2023/0028648 A1
(43) Pub. Date: Jan. 26, 2023

(54) EXPANDABLE STENT HAVING OUTFLOW COMMISSURE POSTS FOR TRANSCATHETER IMPLANTATION OF A CARDIAC VALVE PROSTHESIS

(71) Applicant: Medtronic CV Luxembourg S.a.r.l., Luxembourg (LU)

(72) Inventors: Yas NEUBERGER, Santa Rosa, CA (US); Salvador AVELAR, Santa Rosa, CA (US); Shahnaz JAVANI, Santa Rosa, CA (US); Stuart KARI, Santa Rosa, CA (US); Richard KLEIN, Santa Rosa, CA (US)

(21) Appl. No.: 17/767,758

(22) PCT Filed: Oct. 19, 2020
(86) PCT No.: PCT/EP2020/079407

§ 371 Date: Apr. 8, 2022

Related U.S. Application Data
(60) Provisional application No. 62/923,657, filed on Oct. 21, 2019.

(51) Int. Cl. A61F 2/24

(52) U.S. Cl. CPC A61F 2/2418; A61F 2250/001; A61F 2210/0014; A61F 2250/0018; A61F 2250/0063; A61F 2250/0036; A61F 2250/0037

(57) ABSTRACT

A transcatheter valve prosthesis includes a stent and a prosthetic valve. The stent is mechanically or balloon expandable. The stent has an inflow portion and an outflow portion. The inflow portion includes a plurality of side openings defined by a plurality of crowns and a plurality of struts. The outflow portion has three circumferentially spaced apart commissure posts. The prosthetic valve is disposed within and secured to at least the outflow portion of the stent. The prosthetic valve is configured to block blood flow in one direction to regulate blood flow through a central lumen of the stent. The commissure posts are configured to flex or bend flex radially inwardly to reduce stresses observed during valve loading and thereby improve or increase tissue durability of the prosthetic valve.

### CROSS-REFERENCE TO RELATED APPLICATIONS

This application claims the benefit of U.S. Provisional Patent Application Ser. No. 62/923,657, filed Oct. 21, 2019, which is hereby incorporated by reference in its entirety for all purposes.

### FIELD

The present invention relates to transcatheter valve prostheses that are radially expandable mechanically or by a balloon.

### BACKGROUND

A human heart includes four heart valves that determine the pathway of blood flow through the heart: the mitral valve, the tricuspid valve, the aortic valve, and the pulmonary valve. The mitral and tricuspid valves are atrioventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Recently, flexible prosthetic valves supported by stent structures that can be delivered percutaneously using a catheter-based delivery system have been developed for heart and venous valve replacement. These prosthetic valves may include either self-expanding or balloon-expandable stent structures with valve leaflets attached to the interior of the stent structure. The prosthetic valve can be reduced in diameter, by crimping onto a balloon catheter or by being contained within a sheath component of a delivery catheter, and advanced through the venous or arterial vasculature. Once the prosthetic valve is positioned at the treatment site, for instance within an incompetent native valve, the stent structure may be expanded to hold the prosthetic valve firmly in place.

When designing a prosthetic valve, valve-frame integration and frame mechanical performance often have competing needs or requirements. For example, when attaching the valve to the frame during valve-frame integration, the valve itself needs to be reinforced to the frame at certain locations without hindering mechanical performance of the frame. Embodiments hereof relate to an improved balloon-expandable transcatheter valve prosthesis configured to minimize tradeoffs between the above-described competing needs.

### SUMMARY

According to a first embodiment hereof, the present disclosure provides a transcatheter valve prosthesis including a stent and a prosthetic valve. The stent has a crimped configuration for delivery within a vasculature and an expanded configuration for deployment within a native heart valve. The stent is mechanically or balloon expandable. The stent has an inflow portion and an outflow portion. The inflow portion is formed proximate to an inflow end of the tubular stent, and includes a plurality of crowns and a plurality of struts, with each crown being formed between a pair of opposing struts. A plurality of side openings are defined by the plurality of crowns and the plurality of struts. The outflow portion is formed proximate to an outflow end of the tubular stent and is coupled to the inflow portion. The outflow portion has exactly three commissure posts, each commissure post longitudinally extending from a crown of the inflow portion and the three commissure posts being circumferentially spaced apart. A thickness of each commissure post varies along a length thereof such that a first end is relatively thicker than a second end, the first end being coupled to the crown of the inflow portion. The prosthetic valve is disposed within and secured to at least the outflow portion of the tubular stent. The prosthetic valve is configured to block blood flow in one direction to regulate blood flow through a central lumen of the stent.

In an aspect of the first embodiment, and in combination with any other aspects herein, the disclosure provides the prosthetic valve includes three leaflets and three commissures, each commissure being formed by attached adjacent lateral ends of an adjoining pair of the three leaflets, and the three commissure posts are aligned with and attached to a respective commissure of the three leaflets of the prosthetic valve.

In an aspect of the first embodiment, and in combination with any other aspects herein, the disclosure provides each commissure post is a planar bar.

In an aspect of the first embodiment, and in combination with any other aspects herein, the disclosure provides the thickness of each commissure post is configured to permit each commissure post to flex radially inward during loading of the transcatheter valve prosthesis.

In an aspect of the first embodiment, and in combination with any other aspects herein, the disclosure provides each strut of the inflow portion has a thickness along a length thereof and the thickness of each commissure post at the first end thereof is not greater than the thickness of the strut of the inflow portion.

In an aspect of the first embodiment, and in combination with any other aspects herein, the disclosure provides each commissure post has a pre-set curve such that the second end is disposed radially inward relative to the first end. In an embodiment, the second end of each commissure post is disposed between 1 and 2 mm radially inward relative to the first end.

In an aspect of the first embodiment, and in combination with any other aspects herein, the disclosure provides each strut of the inflow portion has a first width along a length thereof and each commissure post has a second width along a length thereof, the first width being less than the second width.

In an aspect of the first embodiment, and in combination with any other aspects herein, the disclosure provides the inflow portion is formed from a first material and each commissure post of the outflow portion is formed from a second material, the first material being different than the second material. In an embodiment, the first material is plastically deformable and the second material is superelastic. In an embodiment, the second material is Nitinol.

In an aspect of the first embodiment, and in combination with any other aspects herein, the disclosure provides the inflow end of the stent has a total of twelve endmost inflow crowns.

In an aspect of the first embodiment, and in combination with any other aspects herein, the disclosure provides the outflow portion further includes a plurality of axial struts longitudinally extending from a crown of the inflow portion, and at least one axial strut is disposed between circumferentially adjacent commissure posts.

In an aspect of the first embodiment, and in combination with any other aspects herein, the disclosure provides the outflow portion includes exactly six axial frame members, and three of the six axial frame members are the commissure posts and three of the six axial frame members are axial struts, each of the axial struts being disposed between circumferentially adjacent commissure posts.

In an aspect of the first embodiment, and in combination with any other aspects herein, the disclosure provides the inflow portion includes at least three rows of a plurality of struts and crowns, and the at least three rows of the inflow portion are formed between an inflow end of the commissure posts and an inflow end of the stent. In an embodiment, the inflow portion includes exactly three rows of a plurality of struts and crowns.

According to a second embodiment hereof, the present disclosure provides a transcatheter valve prosthesis including a stent and a prosthetic valve. The stent has a crimped configuration for delivery within a vasculature and an expanded configuration for deployment within a native heart valve. The stent is mechanically or balloon expandable. The stent has an inflow portion and an outflow portion. The inflow portion is formed proximate to an inflow end of the stent, and includes a plurality of crowns and a plurality of struts, with each crown being formed between a pair of opposing struts. A plurality of side openings are defined by the plurality of crowns and the plurality of struts. The outflow portion is formed proximate to an outflow end of the stent and is coupled to the inflow portion. The outflow portion has exactly three commissure posts, each commissure post longitudinally extending from a crown of the inflow portion and the three commissure posts being circumferentially spaced apart. Each commissure post has a length that is greater than a length of each strut of the inflow portion, each commissure post has a thickness along the length thereof that is less than a thickness of each strut of the inflow portion along the length thereof, and each commissure post has a width that is greater than a width of each strut of the inflow portion. The prosthetic valve is disposed within and secured to at least the outflow portion of the stent. The prosthetic valve is configured to block blood flow in one direction to regulate blood flow through a central lumen of the stent.

In an aspect of the second embodiment, and in combination with any other aspects herein, the disclosure provides the prosthetic valve includes three leaflets and three commissures, each commissure being formed by attached adjacent lateral ends of an adjoining pair of the three leaflets, and the three commissure posts are aligned with and attached to a respective commissure of the three leaflets of the prosthetic valve.

In an aspect of the second embodiment, and in combination with any other aspects herein, the disclosure provides each commissure post is a planar bar.

In an aspect of the second embodiment, and in combination with any other aspects herein, the disclosure provides each commissure post has a strength that is greater than a strength of each strut of the inflow portion.

In an aspect of the second embodiment, and in combination with any other aspects herein, the disclosure provides the inflow portion is formed from a first material and each commissure post of the outflow portion is formed from a second material, the first material being different than the second material. In an embodiment, the first material is plastically deformable and the second material is superelastic. In an embodiment, the second material is Nitinol.

In an aspect of the second embodiment, and in combination with any other aspects herein, the disclosure provides the inflow end of the stent has a total of twelve endmost inflow crowns.

In an aspect of the second embodiment, and in combination with any other aspects herein, the disclosure provides the outflow portion further includes a plurality of axial struts longitudinally extending from a crown of the inflow portion, and at least one axial strut is disposed between circumferentially adjacent commissure posts.

In an aspect of the second embodiment, and in combination with any other aspects herein, the disclosure provides the outflow portion includes exactly six axial frame members, and three of the six axial frame members are the commissure posts and three of the six axial frame members are axial struts, each of the axial struts being disposed between circumferentially adjacent commissure posts.

In an aspect of the second embodiment, and in combination with any other aspects herein, the disclosure provides the inflow portion includes at least three rows of a plurality of struts and crowns, the at least three rows of the inflow portion are formed between an inflow end of the commissure posts and an inflow end of the stent. In an embodiment, the inflow portion includes exactly three rows of a plurality of struts and crowns.

According to a third embodiment hereof, the present disclosure provides a transcatheter valve prosthesis including a stent and a prosthetic valve. The stent has a crimped configuration for delivery within a vasculature and an expanded configuration for deployment within a native heart valve. The stent is mechanically or balloon expandable. The stent has an inflow portion and an outflow portion. The inflow portion is formed proximate to an inflow end of the tubular stent, and includes a plurality of crowns and a plurality of struts, with each crown being formed between a pair of opposing struts. A plurality of side openings are defined by the plurality of crowns and the plurality of struts. The inflow portion is formed from a first material. The outflow portion is formed proximate to an outflow end of the stent and is coupled to the inflow portion. The outflow portion has exactly three commissure posts, each commissure post longitudinally extending from a crown of the inflow portion and the three commissure posts being circumferentially spaced apart. Each commissure post is formed from a second material different than the first material. A prosthetic valve disposed within and secured to at least the outflow portion of the stent, the prosthetic valve being configured to block blood flow in one direction to regulate blood flow through a central lumen of the stent.

In an aspect of the third embodiment, and in combination with any other aspects herein, the disclosure provides the prosthetic valve includes three leaflets and three commissures, each commissure being formed by attached adjacent lateral ends of an adjoining pair of the three leaflets, and the three commissure posts are aligned with and attached to a respective commissure of the three leaflets of the prosthetic valve.

In an aspect of the third embodiment, and in combination with any other aspects herein, the disclosure provides each commissure post is a planar bar.

In an aspect of the third embodiment, and in combination with any other aspects herein, the disclosure provides each commissure post has a length that is greater than a length of each strut of the inflow portion, each commissure post has a thickness along the length thereof that is less than a thickness of each strut of the inflow portion along the length thereof, and each commissure post has a width that is greater than a width of each strut of the inflow portion.

In an aspect of the third embodiment, and in combination with any other aspects herein, the disclosure provides the first material is plastically deformable and the second material is superelastic. In an embodiment, the second material is Nitinol.

In an aspect of the third embodiment, and in combination with any other aspects herein, the disclosure provides the inflow end of the stent has a total of twelve endmost inflow crowns.

In an aspect of the third embodiment, and in combination with any other aspects herein, the disclosure provides the outflow portion further includes a plurality of axial struts longitudinally extending from a crown of the inflow portion, and at least one axial strut is disposed between circumferentially adjacent commissure posts.

In an aspect of the third embodiment, and in combination with any other aspects herein, the disclosure provides the outflow portion includes exactly six axial frame members, and three of the six axial frame members are the commissure posts and three of the six axial frame members are axial struts, each of the axial struts being disposed between circumferentially adjacent commissure posts.

In an aspect of the third embodiment, and in combination with any other aspects herein, the disclosure provides the inflow portion includes at least three rows of a plurality of struts and crowns, and the at least three rows of the inflow portion are formed between an inflow end of the commissure posts and an inflow end of the stent. In an embodiment, the inflow portion includes exactly three rows of a plurality of struts and crowns.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective side view of a transcatheter valve prosthesis according to an embodiment hereof, wherein the transcatheter valve prosthesis is in an expanded configuration.

FIG. 1A is an end view illustration of the transcatheter valve prosthesis of FIG. 1.

FIG. 2 is a side view of a transcatheter valve prosthesis according to another embodiment hereof, wherein the transcatheter valve prosthesis is relatively longer than the transcatheter valve prosthesis of FIG. 1 and is shown in an expanded configuration.

FIG. 3 is a side view illustration of the transcatheter valve prosthesis of FIG. 1 implanted within a native aortic valve annulus.

FIG. 4 is a perspective view of the stent of the transcatheter valve prosthesis of FIG. 1, wherein the stent is in the expanded configuration.

FIG. 5 is a side view of the stent of the transcatheter valve prosthesis of FIG. 1, wherein the stent is in a non-expanded or crimped configuration.

FIG. 6 is a side view of the stent of the transcatheter valve prosthesis of FIG. 1, wherein the stent is in the expanded configuration.

FIG. 6A is an enlarged side view of a single cell or side opening of an inflow portion of the stent of the transcatheter valve prosthesis of FIG. 1, wherein the stent is in the expanded configuration.

FIG. 7 is an end view of an inflow end of the stent of the transcatheter valve prosthesis of FIG. 1.

FIG. 8 is an end view of an outflow end of the stent of the transcatheter valve prosthesis of FIG. 1.

FIG. 9 is a perspective view of a stent of a transcatheter valve prosthesis according to another embodiment hereof, wherein the stent is in the expanded configuration.

FIG. 10 is a side view of the stent of the transcatheter valve prosthesis of FIG. 9, wherein the stent is in a non-expanded or crimped configuration.

FIG. 11 is a front view of a commissure post of the stent of the transcatheter valve prosthesis of FIG. 1, wherein the commissure post has a variable wall thickness.

FIG. 12 is a side view of the commissure post of FIG. 11.

FIG. 13 is a side view of the commissure post of the stent of the transcatheter valve prosthesis of FIG. 11 according to an embodiment hereof

FIG. 14 is a side view of the commissure post of the stent of the transcatheter valve prosthesis of FIG. 11 according to an embodiment hereof

FIG. 15 is a side view of the commissure post of FIG. 14, wherein bending action of the commissure post is illustrated.

FIG. 16 is a front view of a commissure post of a stent according to another embodiment hereof, wherein the commissure post is pre-set in a curved configuration.

FIG. 17 is a side view of the commissure post of FIG. 16.

FIG. 18 is a side view of a commissure post of a stent according to another embodiment hereof, wherein the commissure post is pre-set in a curved configuration.

FIG. 19 is a perspective view of a stent of a transcatheter valve prosthesis according to another embodiment hereof, wherein the stent is in the expanded configuration and the commissure posts thereof are relatively longer, wider, and thinner than the struts of an inflow portion of the stent.

FIG. 20 is a side view of the stent of the transcatheter valve prosthesis of FIG. 19, wherein the stent is in a non-expanded or crimped configuration.

FIG. 21 is a front view of a commissure post of the stent of the transcatheter valve prosthesis of FIG. 19 according to an embodiment hereof.

FIG. 22 is a side view of FIG. 21.

FIG. 23 is a side view of FIG. 21, wherein bending action of the commissure post is illustrated.

FIG. 24 is a perspective view of a stent of a transcatheter valve prosthesis according to another embodiment hereof, wherein the stent is in the expanded configuration and the commissure posts thereof are formed from a superelastic material.

FIG. 25 is a side view of the stent of the transcatheter valve prosthesis of FIG. 24, wherein the stent is in a non-expanded or crimped configuration.

### DETAILED DESCRIPTION

Specific embodiments of the present invention are now described with reference to the figures, wherein like reference numbers indicate identical or functionally similar elements. The terms “distal” and “proximal”, when used in the following description to refer to a native vessel, native valve, or a device to be implanted into a native vessel or native valve, such as a heart valve prosthesis, are with reference to the direction of blood flow. Thus, “distal” and “distally” refer to positions in a downstream direction with respect to the direction of blood flow and the terms “proximal” and “proximally” refer to positions in an upstream direction with respect to the direction of blood flow.

The following detailed description is merely exemplary in nature and is not intended to limit the invention or the application and uses of the invention. Although the description of the invention is in the context of treatment of an aortic heart valve, the invention may also be used where it is deemed useful in other valved intraluminal sites that are not in the heart. For example, the present invention may be applied to other heart valves or venous valves as well. Furthermore, there is no intention to be bound by any expressed or implied theory presented in the preceding technical field, background, brief summary or the following detailed description.

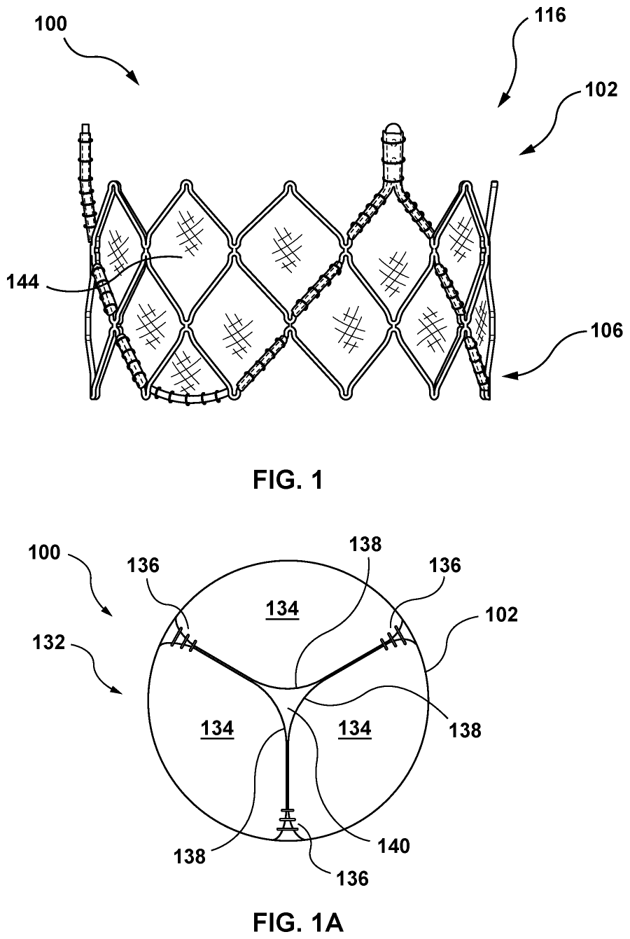

Embodiments hereof relate to a transcatheter valve prosthesis 100 having a radially-expandable stent 102 and a prosthetic valve 132. The stent 102 is generally tubular, and is mechanically or balloon expandable, having a crimped configuration for delivery within a vasculature and an expanded configuration for deployment within a native heart valve. FIG. 1 is a perspective side view of the transcatheter valve prosthesis 100 in the expanded configuration, while FIG. 1A is an end view illustration of the transcatheter valve prosthesis 100. When the transcatheter valve prosthesis 100 is deployed within the valve annulus of a native heart valve, the stent 102 of the transcatheter valve prosthesis 100 is configured to be radially expanded within native valve leaflets of the patient's defective valve, to thereby retain the native valve leaflets in a permanently open state. In embodiments hereof, the transcatheter valve prosthesis 100 is configured for replacement for an aortic valve such that an inflow end 106 of the transcatheter valve prosthesis 100 extends into and anchors within the aortic annulus of a patient's left ventricle, while an outflow end 116 of the transcatheter valve prosthesis 100 is positioned within the aortic sinuses.

The stent 102 of the transcatheter valve prosthesis 100 may be a unitary frame or scaffold that supports the prosthetic valve 132 including one or more valve leaflets 134 within the interior of the stent 102. The prosthetic valve 132 is configured to block flow in one direction to regulate flow there-through via the valve leaflets 134 that may form a bicuspid or tricuspid replacement valve. FIG. 1A is an end view of FIG. 1 taken from the outflow end 116 of the prosthesis and illustrates an exemplary tricuspid valve having three valve leaflets 134, although a bicuspid leaflet configuration may alternatively be used in embodiments hereof. More particularly, as the transcatheter valve prosthesis 100 is configured for placement within a native aortic valve having three leaflets, the prosthetic valve 132 may include three valve leaflets 134. However, the transcatheter valve prosthesis 100 is not required to have the same number of leaflets as the native valve. If the transcatheter valve prosthesis 100 is alternatively configured for placement within a native valve having two leaflets such as the mitral valve, the prosthetic valve 132 may include two or three valve leaflets. The valve leaflets 134 may be attached to a graft material 144 which encloses or lines a portion of the stent 102 as would be known to one of ordinary skill in the art of prosthetic tissue valve construction. The valve leaflets 134 are sutured or otherwise securely and sealingly attached along their bases to the interior surface of the graft material 144, or otherwise attached to the stent 102. Adjoining pairs of leaflets are attached to one another at their lateral ends to form commissures 136, with free edges 138 of the valve leaflets 134 forming coaptation edges that meet in area of coaptation 140. The commissures 136 of the leaflets are aligned with and attached to the commissure posts 126A of the stent 102, which will be described in more detail herein

The valve leaflets 134 may be made of pericardial material; however, the valve leaflets 134 may instead be made of another material. In an embodiment, the valve leaflets 134 are made from bovine pericardial tissue. Natural tissue for the valve leaflets 134 may be obtained from, for example, heart valves, aortic roots, aortic walls, aortic leaflets, pericardial tissue, such as pericardial patches, bypass grafts, blood vessels, intestinal submucosal tissue, umbilical tissue and the like from humans or animals. Synthetic materials suitable for use as the valve leaflets 134 include DACRON® polyester commercially available from Invista North America S.A.R.L. of Wilmington, Del., other cloth materials, nylon blends, polymeric materials, and vacuum deposition nitinol fabricated materials. One polymeric material from which the leaflets can be made is an ultra-high molecular weight polyethylene material commercially available under the trade designation DYNEEMA from Royal DSM of the Netherlands. With certain leaflet materials, it may be desirable to coat one or both sides of the leaflet with a material that will prevent or minimize overgrowth. It is further desirable that the leaflet material is durable and not subject to stretching, deforming, or fatigue.

Graft material 144 may enclose or line the stent 102 as would be known to one of ordinary skill in the art of prosthetic tissue valve construction. Graft material 144 may be a natural or biological material such as pericardium or another membranous tissue such as intestinal submucosa. Alternatively, graft material 144 may be a low-porosity woven fabric, such as polyester, Dacron fabric, or PTFE. In one embodiment, graft material 144 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example.

As previously stated, the stent 102 is mechanically or balloon-expandable as would be understood by one of ordinary skill in the art. As such, the stent 102 is made from a plastically deformable material such that when expanded by a dilatation balloon or other mechanical expansion device, the stent 102 maintains its radially expanded configuration. The stent 102 may be formed from stainless steel or other suitable metal, such as platinum iridium, cobalt chromium alloys such as MP35N, or various types of polymers or other materials known to those skilled in the art, including said materials coated with various surface deposits to improve clinical functionality. The stent 102 is configured to be rigid such that it does not deflect or move when subjected to in-vivo forces, or such that deflection or movement is minimized when subjected to in-vivo forces. In an embodiment, the radial stiffness (i.e., a measurement of how much the tubular stent 102 deflects when subjected to in-vivo forces) of the tubular stent 102 is between 80 N/m and 120 N/m, and the radial stiffness of the stent 102 scaled across the deployed height thereof is approximately 5 N/mm². In an embodiment, the radial stiffness of the tubular stent 102 is greater than 100 N/m. Further, in an embodiment, the device recoil (i.e., a measurement of how much the stent 102 relaxes after balloon deployment) is below 15% and the approximately recoil after deployment is between 1 mm and 2 mm. Further, in an embodiment, the device crush or yield (i.e., the radial force at which the tubular stent 102 yields) is approximately 200 N.

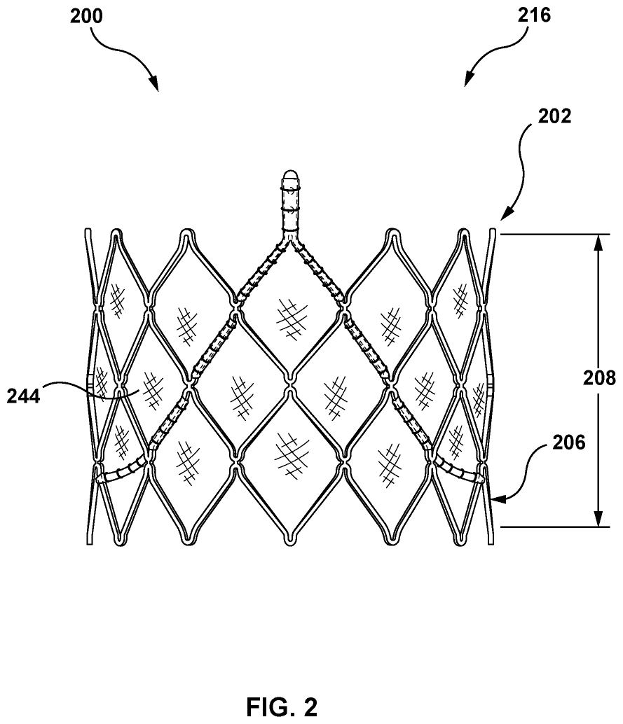

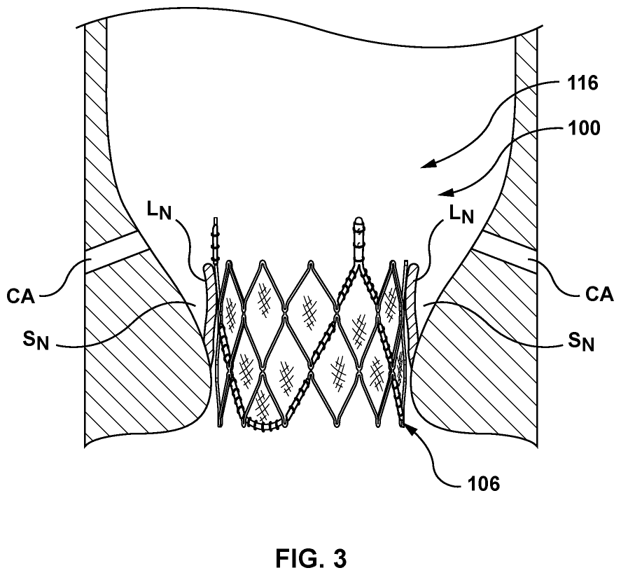

Delivery of the transcatheter valve prosthesis 100 may be accomplished via a percutaneous transfemoral approach or a transapical approach directly through the apex of the heart via a thoracotomy, or may be positioned within the desired area of the heart via different delivery methods known in the art for accessing heart valves. The transcatheter valve prosthesis 100 has a crossing profile of between 15-30 Fr, the crossing profile being defined as the outside diameter (OD) of the transcatheter valve prosthesis 100 after it is crimped onto a delivery catheter and allowed to recoil from the crimping action. During delivery, the transcatheter valve prosthesis 100 remains compressed until it reaches a target diseased native heart valve, at which time a balloon of a balloon catheter is inflated or other mechanical expansion device is expanded in order to radially expand the transcatheter valve prosthesis 100 in situ. The delivery catheter is then removed and the transcatheter valve prosthesis 100 remains deployed within the native target heart valve. FIG. 3 illustrates the transcatheter valve prosthesis 100 implanted in situ within a native aortic valve annulus, which is shown in section, having native leaflets LN and corresponding native sinuses SN. FIG. 3 also illustrates placement of the coronary arteries CA. The transcatheter valve prosthesis 100 is configured for intra-annular placement within a native aortic valve. More particularly, the inflow end 106 of the transcatheter valve prosthesis 100 extends into and anchors within the aortic annulus of a patient's left ventricle, while the outflow end 116 of the transcatheter valve prosthesis 100 is positioned within the aortic sinuses, with no portion of the transcatheter valve prosthesis 100 extending into the patient's ascending aorta. When the transcatheter valve prosthesis 100 is deployed within the valve annulus of a native heart valve, the stent 102 is configured to be expanded within native valve leaflets LN of the patient's defective valve, to thereby retain the native valve leaflets in a permanently open state. A height or length of the stent 102 in the expanded configuration is between 12 and 24 mm, the height being measured from the most proximal part thereof (endmost inflow crowns 110A, which will be described in more detail herein) to the most distal part thereof (second ends 130A of commissure posts 126A, which will be described in more detail herein). In an embodiment hereof, a height or length of the stent 102 in the expanded configuration is between 18 and 24 mm. For example, in an embodiment the stent 102 has diameter of between 21-24 mm and a height of 19 mm. In another embodiment, the stent 102 has diameter of between 24-27 mm and a height of 21 mm. In yet another embodiment, the stent 102 has diameter of between 27-30 mm and a height of 23 mm.

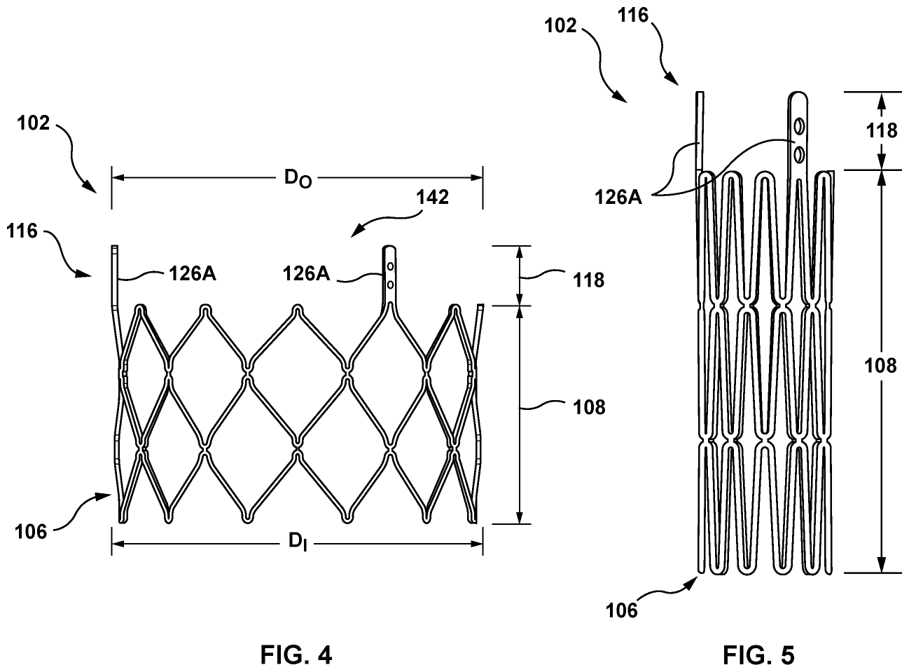

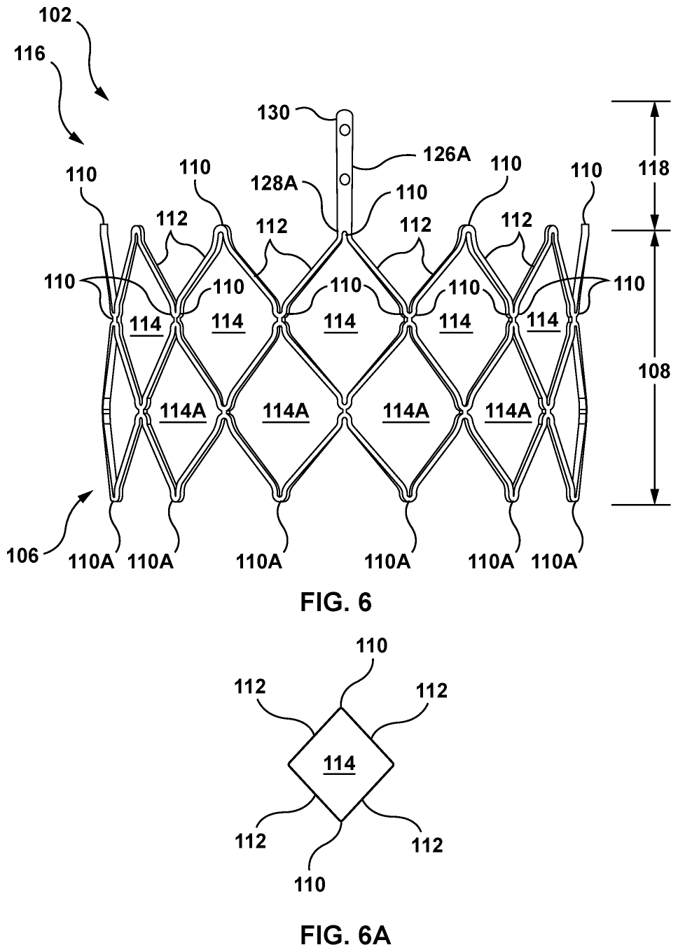

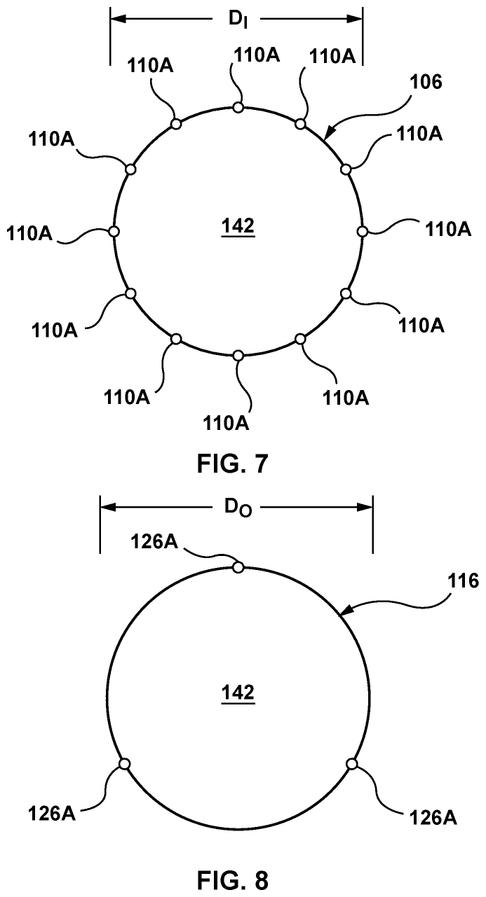

With reference to FIGS. 4-8, the stent 102 will now be described in more detail. The stent 102 has an expanded configuration, which is shown in the perspective and side views of FIGS. 4 and 6, respectively, and a non-expanded or crimped configuration, which is shown in the side view of FIG. 5. Non-expanded or crimped configuration as used herein refers to the configuration of the stent 102 after crimping onto a catheter, e.g., after crimping onto a balloon of a balloon catheter, for delivery. FIG. 7 is an end view of the inflow end 106 of the stent 102, while FIG. 8 is an end view of the outflow end 116 of the stent 102. The stent 102 has an inflow portion 108 and an outflow portion 118. The stent 102 is a tubular component defining a central lumen or passageway 142, and further defines the inflow or proximal end 106 and the outflow or distal end 116 of the transcatheter valve prosthesis 100. The inflow portion 108 forms the generally tubular shape of the stent 102 and the outflow portion 118 includes three commissure bars 126A longitudinally extending from the inflow portion 108, as will be described in more detail herein. When expanded, a diameter D₁ of the inflow end 106 of the stent 102 is the same as a diameter D₀ of the outflow end 116 of the stent 102. In an embodiment, the diameters D₁ and D₀ may range between 18 and 30 mm in order to accommodate dimensions of the native valve anatomy. Stated another way, it may be desirable for the transcatheter valve prosthesis 100 to be available in varying size increments to accommodate varying diameters or sizes of a patient's native annulus. The stent 102 may be formed by a laser-cut manufacturing method and/or another conventional stent forming method as would be understood by one of ordinary skill in the art. The cross-section of the stent 102 may be circular, ellipsoidal, rectangular, hexagonal, square, or other polygonal shape, although at present it is believed that circular or ellipsoidal may be preferable with the transcatheter valve prosthesis 100 being provided for replacement of an aortic valve.

The inflow portion 108 is formed proximate to the inflow end 106 of the stent 102. The inflow portion 108 includes a plurality of crowns 110 and a plurality of struts 112 with each crown 110 being formed between a pair of opposing struts 112. Each crown 110 is a curved segment or bend extending between opposing struts 112. The inflow portion 108 is tubular, with a plurality of cells or side openings 114 being defined by the plurality of crowns 110 and the plurality of struts 112. In an embodiment, the plurality of side openings 114 may be diamond-shaped. More particularly, as best shown in FIG. 6A which is a side view of a single side opening 114 of the inflow portion 108 of the stent 102, each side opening 114 is formed by two pairs of opposing crowns 110 and four struts 112 therebetween. Each side opening 114 is symmetrical for easier integration with the prosthetic valve 132. A series of endmost inflow side openings 114A and a series of endmost inflow crowns 110A are formed at the inflow end 106 of the stent 102. The inflow end 106 of the stent 102 has a total of twelve endmost inflow crowns 110A, as best shown in the end view of FIG. 7.

In an embodiment, the inflow portion 108 includes exactly three rows of struts 112 and crowns 110 longitudinally between the commissure bars 126A and the inflow end 106 of the stent 102. However, the length or height of the inflow portion 108 may vary from that depicted herein in order to accommodate dimensions of the native valve anatomy. For example, in another embodiment hereof as shown in FIG. 2, a transcatheter valve prosthesis 200 is shown that is relatively longer than the transcatheter valve prosthesis 100. More particularly, the transcatheter valve prosthesis 200 includes a stent 202 having graft material 244 which encloses or lines a portion of the stent 202 as would be known to one of ordinary skill in the art of prosthetic tissue valve construction. The stent 202 is a tubular component that defines an inflow end 206 and an outflow end 216 of the transcatheter valve prosthesis 200. An inflow portion 208 of the stent 202 is relatively longer than the inflow portion 108 of the stent 102 so that the overall length or height of the transcatheter valve prosthesis 200 may be relatively increased to accommodate dimensions of the native valve anatomy. For example, a height or length of the stent 202 in the expanded configuration is between 18-24 mm. In the embodiment of FIG. 2, the inflow portion 208 includes exactly four rows of struts and crowns longitudinally between commissure bars 226A and the inflow end 206 of the stent 202.

The outflow portion 118 is formed proximate to the outflow end 116 of the stent 102. As previously described, the outflow portion 118 includes three commissure posts 126A that longitudinally or axially extend from the inflow portion 108 and are substantially parallel to the central longitudinal axis of the stent 102. Each commissure post 126A is a relatively stiff, axial segment or planar bar having a first end 128A connected to a crown 110 of the inflow portion 108 and an unattached or free second end 130A. The three commissure posts 126A are circumferentially spaced apart and aligned with and attached to a respective commissure of the three leaflets of the prosthetic valve. The prosthetic valve 132 is disposed within and secured to at least the outflow portion 118 of the stent 102 at the commissure posts 126A. In addition, the prosthetic valve 132 may also be disposed within and secured to the inflow portion 108 of the stent 102. The three commissure posts 126A aid in valve alignment and coaptation. More particularly, the three commissure posts 126A reinforce or strengthen the commissure region of the prosthetic valve 132 by shaping the leaflets 134 and supporting the leaflets 134 during opening and closing thereof, and thus provide more reliable leaflet coaptation.

In the embodiment depicted in FIGS. 1-8, the three commissure posts 126A are the only structures formed at the outflow end 118 of the stent 102. Stated another way, the three commissure posts 126A are the only structures distal to the distalmost crowns 110 of the inflow portion 108. The configuration of the stent 102 maximizes access to the coronary arteries because the commissure posts 126A are the only structures in the vicinity of the coronary arteries at the outflow portion 118 of the stent 102. It is very improbable that the right coronary artery and/or the left main coronary artery will be blocked or jailed by the commissure posts 126A, and thus there will be clear access to the coronary arteries via a coronary guide catheter once the transcatheter valve prosthesis 100 is deployed in situ. In addition, with the elimination of any outflow crowns at the outflow portion 118 of the stent 102, the overall height of the stent 102 is reduced relative to a stent having outflow crowns formed distal to the commissure posts 126A. A shorter overall height minimizes interaction with aortic anatomy, thereby resulting in less vessel trauma or valve deformation.

In another embodiment hereof depicted in FIGS. 9 and 10, in addition to the three commissure posts 126A, an outflow portion 918 of a stent 902 may also include axial struts 126B that are disposed circumferentially between adjacent commissure posts 126A. The stent 902 has an expanded configuration, which is shown in the side view of FIG. 9, and a non-expanded or crimped configuration, which is shown in the side view of FIG. 10. Each axial strut 126B is also a relatively stiff, axial segment or planar bar having a first end 128B connected to a crown 110 of the inflow portion 108 and an unattached or free second end 130B. Similar to the commissure posts 126A, each axial strut 126B longitudinally extends from a crown 110 of the inflow portion 108 and is substantially parallel to the central longitudinal axis of the stent 902. However, unlike the commissure posts 126A, the axial struts 126B are not configured to align with and attach to a respective commissure of the three leaflets of the prosthetic valve. The axial struts 126B and the commissure posts 126A are herein referred to collectively as axial frame members 126. In an embodiment, the outflow portion 918 includes up to six axial frame members 126, with three of the axial frame members 126 being the commissure posts 126A and three of the axial frame members 126 being axial struts 126B that are disposed circumferentially between adjacent commissure posts 126A. The axial frame members 126 (i.e., the commissure posts 126A and the axial struts 126B collectively) minimize crossing profile of the transcatheter valve prosthesis while maximizing symmetrical cell expansion. Symmetrical cell expansion ensures that the stent 102 crimps well onto a balloon of a balloon catheter for delivery. Poor crimp quality may lead to portions of a stent overlapping when crimped, which in turn may cause tissue damage to the valve leaflets of the prosthetic valve during the crimping process.

The configuration of the stent 902 also provides good access to the coronary arteries because the axial frame members 126 are the only structures in the vicinity of the coronary arteries at the outflow portion 918 of the stent 902. Even with the addition of the axial struts 126B, it is very improbable that the right coronary artery and/or the left main coronary artery will be blocked or jailed by the axial frame members 126, and thus there will be clear access to the coronary arteries via a coronary guide catheter once the transcatheter valve prosthesis is deployed in situ. In addition, with the elimination of any outflow crowns at the outflow portion 918 of the stent 902, the overall height of the stent 902 is reduced relative to a stent having outflow crowns formed distal to the axial frame members 126. A shorter overall height minimizes interaction with aortic anatomy, thereby resulting in less vessel trauma or valve deformation.

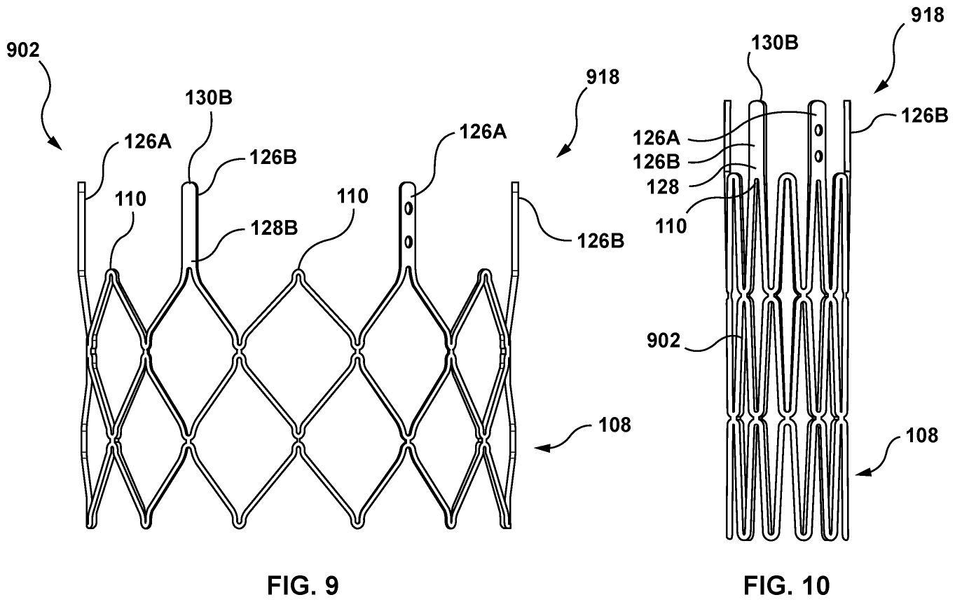

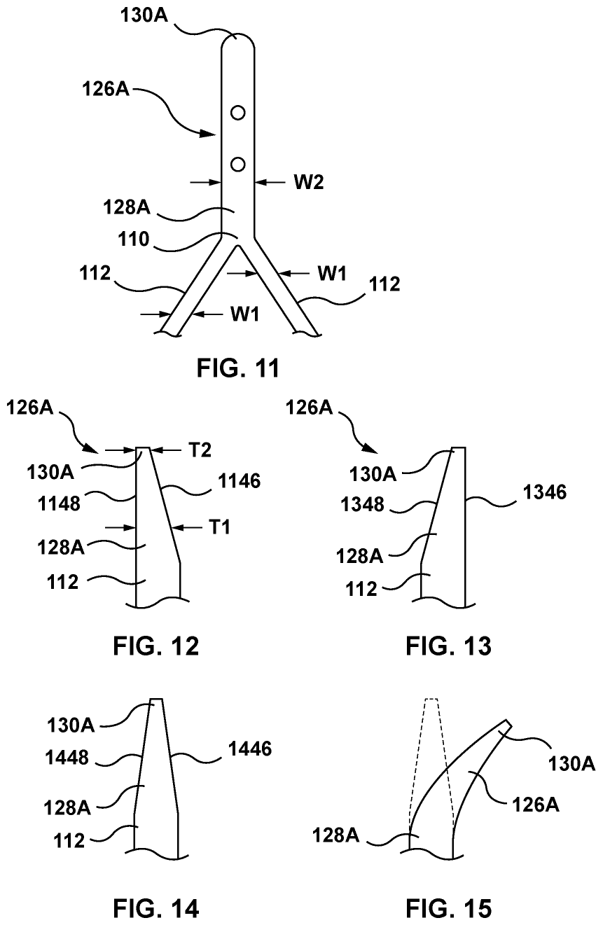

Each commissure post 126A of the stent 102 is configured to flex radially inward during loading of the transcatheter valve prosthesis 100. More particularly, with reference to FIGS. 11-15, a thickness of each commissure post 126A varies along a length thereof such that the first end 128A (which is coupled to or extends from the crown 110 of the inflow portion 108) is relatively thicker than the second end 130A. The decrease or taper of the thickness of the commissure posts 126A at the outflow portion 118 of the tubular stent 102 configures the commissure posts 126A to flex slightly radially inwardly to reduce stresses observed during valve loading. By flexing or bending radially inward, the commissure posts 126A improve or increase tissue durability of the valve leaflets 134 because the strains experienced during valve loading are transferred to the commissure posts 126A.

More particularly, as compared to self-expanding valve stents, balloon expandable valve stents are stiffer and stronger but therefore may place more stress on the valve leaflets 134 attached to the stent 102. The valve leaflets 134, which are often formed from tissue, are more durable when the portion of the stent to which they are attached is more flexible, but such stent flexibility may be detrimental to stent fatigue. As such, the variable thickness of the commissure posts 126A achieves a balance between stent durability and tissue durability. Therefore, by varying a wall thickness of the commissure post 126A in the radial direction to tune the flexure of the commissure post 126A, the stent 102 maintains its strength and durability while permitting the commissure posts 126A to flex inward to increase tissue durability. Stated another way, the variable thickness of the commissure posts 126A extends the useable life of the balloon expandable transcatheter valve prosthesis 100.

More particularly, FIG. 11 is a front view of a commissure post 126A of the stent 102. As shown in FIG. 12, which is a side view of the commissure post 126A according to an embodiment, a wall thickness of the commissure post 126A is varied in the radial direction to tune the flexure of the commissure post 126A. A first thickness T1 at the first end 128A of the commissure post 126A is thicker than a second thickness T2 at the second end 130A of the commissure post 126A. The thickness gradually tapers from the first thickness T1 to the second thickness T2 such that the tip or second end 130A is configured to flex radially inward while the base or first end 128A is thicker at the junction of the crown 110 of the inflow portion 108 to sustain loads. Each stmt 112 of the inflow portion 108 adjacent to the commissure post 126A has a uniform thickness along a full or entire length thereof and the thickness T1 of the commissure post 126A at the first end 128A is not greater than the thickness of the struts 112 of the inflow portion 108. The commissure post 126A is permitted to deflect or bend radially inward in a controlled and predictable manner, as shown in FIG. 15, and this controlled deflection or bending increases tissue durability as described above without sacrificing durability of the stent 102.

Each of the embodiments of FIGS. 12, 13, and 14 illustrate how a wall thickness of the commissure posts 126A may be varied in the radial direction to tune the flexure of the commissure post 126A such that the commissure posts 126A are configured to flex radially inward during loading of the transcatheter valve prosthesis 100. The variable wall thickness of the commissure posts 126A can be shaped or formed by micro-blasting and electropolishing the target surface(s).

In the embodiment of FIGS. 11 and 12, an innermost radial surface 1146 (i.e., the surface in the direction of the central longitudinal axis of the stent 102) of the commissure post 126A is tapered while an outermost radial surface 1148 of the commissure post 126A is flush with an outer surface 1150 of the inflow portion 108. More particularly, the innermost radial surface 1146 of the commissure post 126A tapers radially outward in a direction from the first end 128A to the second end 130A of the commissure post 126A. Stated another way, in this embodiment, the wall thickness of the commissure post 126A is reduced via the innermost radial surface 1146 of the commissure post 126A. In the embodiment of FIG. 13, however, an outermost radial surface 1348 of the commissure post 126A is tapered while an innermost radial surface 1346 of the commissure post 126A is flush with an inner surface 1152 of the inflow portion 108. More particularly, the outermost radial surface 1348 of the commissure post 126A tapers radially inward in a direction from the first end 128A to the second end 130A of the commissure post 126A. Stated another way, in this embodiment, the wall thickness of the commissure post 126A is reduced via the outermost radial surface 1348 of the commissure post 126A. Lastly, in the embodiment of FIG. 14, an innermost radial surface 1446 of the commissure post 126A is tapered radially outward and an outermost radial surface 1448 of the commissure post 126A is tapered radially inward. More particularly, the innermost radial surface 1446 of the commissure post 126A tapers radially outward in a direction from the first end 128A to the second end 130A of the commissure post 126A while the outermost radial surface 1448 of the commissure post 126A tapers radially inward in a direction from the first end 128A to the second end 130A of the commissure post 126A. Stated another way, in this embodiment, the wall thickness of the commissure post 126A is reduced via both the innermost and outermost radial surfaces 1446, 1448 of the commissure post 126A.

In addition to having a variable thickness, the commissure posts 126A also have a width in the circumferential direction that is relatively wider than a width of the struts 112 of the inflow portion 108 adjacent to the commissure post 126A. More particularly, each strut 112 of the inflow portion 108 adjacent to the commissure post 126A has a width W1 along a full or entire length thereof and each commissure post 126A has a width W2 along a full or entire length thereof. Width W1 of the struts 112 is less than the width W2 of the commissure posts 126A. In an embodiment, width W2 is at least two times greater than the width W1 of the struts 112. In another embodiment, width W2 is at least three times greater than the width W1 of the struts 112. In yet another embodiment, width W2 is at least four times greater than the width W1 of the struts 112. The relatively wider commissure posts 126A aid to spread out the load experienced by the commissure posts across a wider area and also results in a cross section for the commissure posts 126A that is amendable to bending radially inwards.

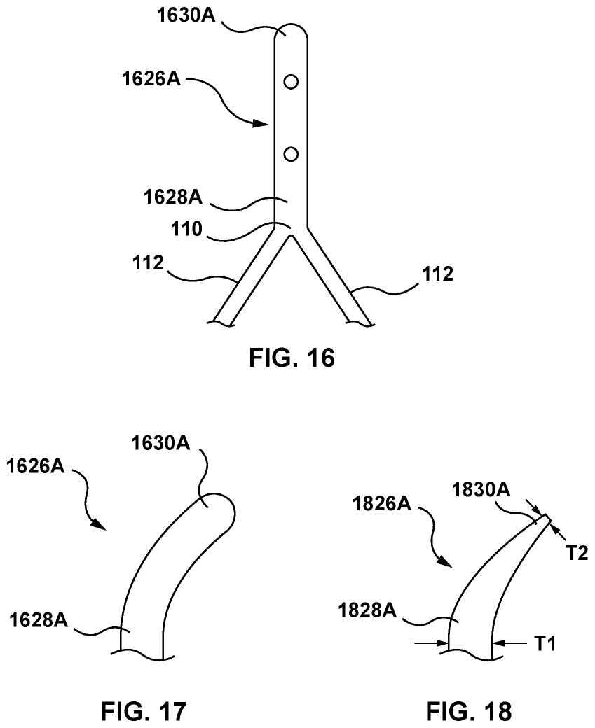

In another embodiment hereof, referring now to FIGS. 16-18, the commissure posts may be pre-formed or pre-set in a curved or bent configuration rather than being configured to flex radially inward upon application of forces during valve loading. More particularly, FIG. 16 is a front view of a commissure post 1626A having a first end 1628A connected to a crown 110 of the inflow portion 108 and an unattached or free second end 1630A. As shown in FIG. 17, which is a side view of the commissure post 1626A, the commissure post 1626A is pre-formed or pre-set in a curved or bent configuration in which the commissure post 1626A curves or bends radially inward. Stated another way, the commissure post 1626A has a pre-set curve such that the second end 1630A is disposed radially inward relative to the first end 1628A. In an embodiment, the second end 1630A of each commissure post 1626A is disposed between 1 and 2 mm radially inward relative to the first end 1630A. In an embodiment, the pre-set curve of the commissure post 1626A may displace the second end 1630A radially inward between 10 and 20 degrees relative to an outer surface of the inflow portion of the tubular stent. The pre-set curve of the commissure posts 1626A function to reduce interaction with the aortic root anatomy and further improve coronary artery access by pulling slightly away from the aortic anatomy. When a transcatheter valve prosthesis having the pre-set curved commissure posts 1626A is crimped onto a balloon for delivery, the commissure posts 1626A may overhang on the balloon so that when the balloon is inflated, the outflow portion having the commissure posts 1626A open last and hence the commissure posts 1626A keep their pre-set curved configuration.

The commissure posts 1626A may have a uniform thickness in the radial direction along a length thereof as shown on FIG. 17, or alternatively may be formed with a variable thickness similar to the embodiments of FIGS. 11-15. More particularly, with reference to another embodiment depicted in FIG. 18, a thickness of a commissure post 1826A varies along a length thereof such that a first end 1828A (which is coupled to the crown 110 of the inflow portion 108) is relatively thicker than a second end 1830A thereof. Similar to the commissure post 1626A, the commissure post 1826A is pre-formed or pre-set in a curved or bent configuration in which the commissure post 1826A curves or bends radially inward. However, in the embodiment of FIG. 18, a first thickness T1 at the first end 1828A of the commissure post 1826A is thicker than a second thickness T2 at the second end 1830A of the commissure post 1826A. The thickness gradually tapers from the first thickness T1 to the second thickness T2 such that the tip or second end 1830A is configured to flex further radially inward during valve loading while the base or first end 1828A is thicker at the junction of the crown 110 of the inflow portion 108 to sustain loads.

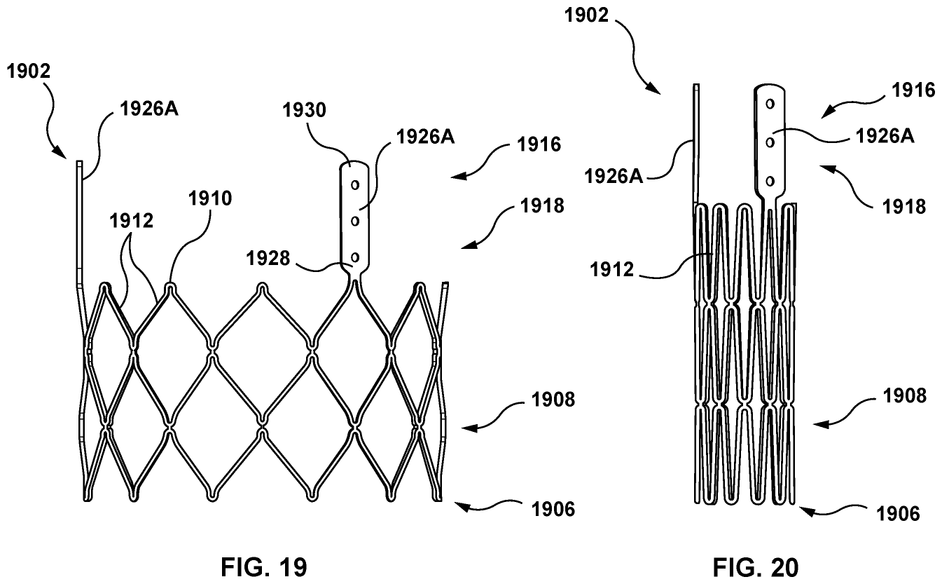

In another embodiment hereof, referring now to FIGS. 19-23, the commissure posts may be relatively longer, wider, and thinner than the struts of the inflow portion such that each commissure post 1926A of a stent 1902 is configured to flex radially inward during loading of the transcatheter valve prosthesis. Similar to the stent 102, the stent 1902 has an expanded configuration, which is shown in the side view of FIG. 19, and a non-expanded or crimped configuration, which is shown in the side view of FIG. 20. Although only the stent 1902 is shown, the stent 1902 is configured to be utilized with a prosthetic valve component to form a transcatheter valve prosthesis similar to the transcatheter valve prosthesis 100 described above. The stent 1902 has an inflow portion 1908 and an outflow portion 1918. The stent 1902 is a tubular component defining a central lumen or passageway (not shown in the side views of FIGS. 19-20), and further defines the inflow or proximal end 1906 and the outflow or distal end 1916 of a transcatheter valve prosthesis. The inflow portion 1908 forms the generally tubular shape of the stent 1902 and the outflow portion 1918 includes three commissure bars 1926A longitudinally extending from the inflow portion 1908, as will be described in more detail herein. The inflow portion 1908 of the stent 1902 has the same structure or configuration as the inflow portion 108 of the stent 102 described above, and therefore details thereof are not repeated. When expanded, a diameter of the inflow end 1906 of the stent 1902 is the same as a diameter of the outflow end 1916 of the stent 1902. In an embodiment, the diameter of the inflow and outflow ends may range between 18 and 30 mm in order to accommodate dimensions of the native valve anatomy. The stent 1902 may be formed by a laser-cut manufacturing method and/or another conventional stent forming method as would be understood by one of ordinary skill in the art. The cross-section of the stent 1902 may be circular, ellipsoidal, rectangular, hexagonal, square, or other polygonal shape, although at present it is believed that circular or ellipsoidal may be preferable with the transcatheter valve prosthesis being provided for replacement of an aortic valve.

The outflow portion 1918 includes three commissure posts 1926A that longitudinally or axially extend from the inflow portion 1908 and are substantially parallel to the central longitudinal axis of the tubular stent 1902. Similar to commissure posts 126A, each commissure post 1926A is a relatively stiff, axial segment or planar bar having a first end 1928 connected to a crown 1910 of the inflow portion 1908 and an unattached or free second end 1930. The three commissure posts 1926A are circumferentially spaced apart and aligned with and attached to a respective commissure of the three leaflets of the prosthetic valve. A prosthetic valve (not shown in FIGS. 19-20), similar to the prosthetic valve 132 described above, is disposed within and secured to at least the outflow portion 1918 of the stent 1902 at the commissure posts 1926A. The three commissure posts 1926A aid in valve alignment and coaptation. More particularly, the three commissure posts 1926A reinforce or strengthen the commissure region of the prosthetic valve by shaping the leaflets thereof and supporting the leaflets during opening and closing thereof, and thus provide more reliable leaflet coaptation.

In the embodiment depicted in FIGS. 19-20, the three commissure posts 1926A are the only structures formed at the outflow end 1918 of the stent 1902. The configuration of the stent 1902 maximizes access to the coronary arteries because the commissure posts 1926A are the only structures in the vicinity of the coronary arteries at the outflow portion 1918 of the stent 1902. It is very improbable that the right coronary artery and/or the left main coronary artery will be blocked or jailed by the commissure posts 1926A, and thus there will be clear access to the coronary arteries via a coronary guide catheter once the transcatheter valve prosthesis is deployed in situ. In addition, with the elimination of any outflow crowns at the outflow portion 1918 of the tent 1902, the overall height of the stent 1902 is reduced relative to a stent having outflow crowns formed distal to the commissure posts 1926A. A shorter overall height minimizes interaction with aortic anatomy, thereby resulting in less vessel trauma or valve deformation. In another embodiment hereof (not shown), in addition to the three commissure posts 1926A, the outflow portion 1918 of the stent 1902 may also include axial struts that are disposed between circumferentially adjacent commissure posts 1926A similar to the axial struts 126B described in the embodiment of FIGS. 9-10.

As previously stated, the commissure posts 1926A may be relatively longer, wider, and thinner than the struts 1912 of the inflow portion 1910 such that each commissure posts 1926A of the stent 1902 is configured to flex radially inward during loading of the transcatheter valve prosthesis. The longer, wider, and thinner commissure posts 1926A are configured to flex slightly radially inwardly to reduce stresses observed during valve loading and thereby improve or increases tissue durability of the valve leaflets attached thereto because the strains experienced during valve loading are transferred to the commissure posts 1926A. More particularly, as compared to self-expanding valve stents, balloon expandable valves stents are stiffer and stronger but therefore may place more stress on the valve leaflets attached thereto attached to the stent 1902. The valve leaflets, which are often formed from tissue, are more durable when the portion of the stent to which they are attached is more flexible, but such stent flexibility may be detrimental to stent fatigue. As such, the longer, wider, and thinner commissure posts 1926A achieve a balance between stent durability and tissue durability because the stent 1902 maintains its strength and durability while permitting the commissure posts 1926A to flex inward to increase tissue durability. Stated another way, the longer, wider, and thinner commissure posts 1926A extend the useable life of the balloon expandable transcatheter valve prosthesis. The dimensions of the longer, wider, and thinner commissure posts 1926A can be accomplished by micro-blasting, bead blasting, electropolishing, or swaging the commissure posts 1926A after the stent 1902 is formed by a laser-cut manufacturing method and/or another conventional stent forming method.

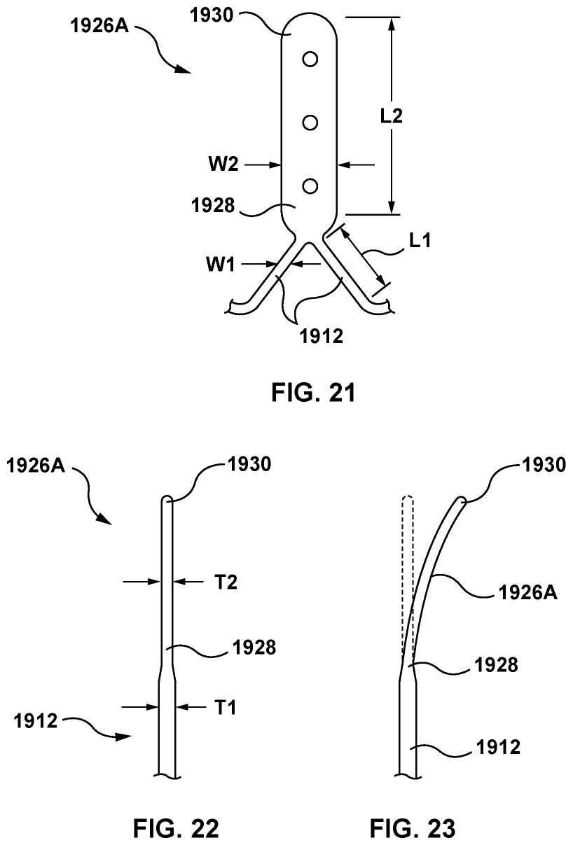

FIG. 21 is a front view of a commissure post 1926A of the stent 1902 and FIG. 22 is a side view of FIG. 21. Each commissure post 1926A has a length that is greater than a length of each strut 1912 of the inflow portion 1908, each commissure post 1926A has a thickness along the length thereof that is less than a thickness of each strut 1912 of the inflow portion 1908 along the length thereof, and each commissure post 1926A has a width that is greater than a width of each strut 1912 of the inflow portion 1908. The commissure post 1926A is permitted to deflect or bend radially inward in a controlled and predictable manner, as shown in FIG. 23, and this controlled deflection or bending increases tissue durability as described above without sacrificing durability of the stent 1902.

More particularly, each strut 1912 of the inflow portion 1908 adjacent to the commissure post 1926A has a uniform thickness T1 along a full or entire length thereof, and each commissure post 1926A has a uniform thickness T2 along a full or entire length thereof. As best shown in the side view of FIG. 22, the thickness T2 of the commissure post 1926A that is less than the thickness T1 of each strut 1912 of the inflow portion 1908. In an embodiment, thickness T1 of the struts 1912 is at least two times greater than the thickness T2 of the commissure posts 1926A. In another embodiment, thickness T1 of the struts 1912 is at least three times greater than the thickness T2 of the commissure posts 1926A. In yet another embodiment, thickness T1 of the struts 1912 is at least four times greater than the thickness T2 of the commissure posts 1926A. The wall thickness T2 of the commissure posts 1926A may be reduced in the radial direction by micro-blasting and electropolishing the target surface(s). As described above with respect to FIGS. 11-15, the surface(s) that may be targeted to reduce the wall thickness T2 of the commissure posts 1926A may be the innermost radial surfaces of the commissure posts 1926A, the outermost radial surfaces of the commissure posts 1926A, or both the innermost and outermost radial surfaces of the commissure posts 1926A.

In addition to having a reduced thickness relative to the adjacent struts 1912, the commissure posts 1926A also have a width W2 in the circumferential direction that is relatively wider than a width W1 of the struts 1912 of the inflow portion 1908 adjacent to the commissure post 9126A. More particularly, each strut 1912 of the inflow portion 1908 adjacent to the commissure post 1926A has a width W1 along a full or entire length thereof and each commissure post 1926A has a width W2 along a full or entire length thereof. Width W1 of the struts 1912 is less than the width W2 of the commissure posts 1926A. In an embodiment, width W2 is at least two times greater than the width W1 of the struts 1912. In another embodiment, width W2 is at least three times greater than the width W1 of the struts 1912. In yet another embodiment, width W2 is at least four times greater than the width W1 of the struts 1912. The relatively wider commissure posts 1926A aid to spread out the load experienced by the commissure posts across a wider area and also results in a cross section for the commissure posts 1926A that is amendable to bending radially inwards.

In addition to having a reduced thickness and an increased width relative to the adjacent struts 1912, the commissure posts 1926A also have a full or entire length L2 in the longitudinal direction that is relatively longer than a full or entire length L1 of the struts 1912 of the inflow portion 1908 adjacent to the commissure post 9126A. More particularly, each strut 1912 of the inflow portion 1908 adjacent to the commissure post 1926A has a length L1 along a length thereof and each commissure post 1926A has a length L2 along a length thereof. Length L1 of the struts 1912 is less than the length L2 of the commissure posts 1926A. In an embodiment, length L2 is at least two times greater than the length L1 of the struts 1912. In another embodiment, length L2 is at least three times greater than the length L1 of the struts 1912. In yet another embodiment, length L2 is at least four times greater than the length L1 of the struts 1912. For example, in an embodiment the length L1 of the struts 1912 may range between 3 mm and 4 mm while the length L2 of the commissure posts 1926A may range between 5 mm and 7 mm. The relatively longer commissure posts 1926A further results in a cross section for the commissure posts 1926A that is amendable to bending radially inwards.

In an embodiment hereof, in addition to being relatively longer, wider, and thinner than struts 1912 of the inflow portion 1910, each commissure posts 1926A also has a greater strength than a strength of each strut 1912 of the inflow portion 1910. The strength of the commissure posts 1926A may be increased via post processing after the stent 1902 is formed by a laser-cut manufacturing method and/or another conventional stent forming method. More particularly, the yield strength of the material of the commissure posts 1926A is increased due to the cold work (swaging) or other post processing methods. The increased yield strength leads to an increase in fatigue resistance. In addition, the tensile strength of the material of the commissure posts 1926A is also increased due to the cold work (swaging) or other post processing methods. For example, in an embodiment, the commissure posts 1926A may undergo swaging and cold work to increase at least the yield strength of the commissure posts 1926A which would in turn increase the fatigue resistance of the commissure posts 1926A. In another embodiment, at least the yield strength of the commissure posts 1926A as well as the struts 1912 of the distalmost row of struts of the inflow portion of the stent 1902 may be increased via post processing after the stent 1902 is formed by a laser-cut manufacturing method and/or another conventional stent forming method. In yet another embodiment, at least the yield strength of the commissure posts 1926A as well as the struts 1912 forming the endmost diamond-shaped openings of the inflow portion of the stent 1902 may be increased via post processing after the stent 1902 is formed by a laser-cut manufacturing method and/or another conventional stent forming method. Stated another way for this embodiment, at least the yield strength of the commissure posts 1926A as well as the struts 1912 of the two distalmost rows of struts of the inflow portion of the stent 1902 may be increased via post processing after the stent 1902 is formed by a laser-cut manufacturing method and/or another conventional stent forming method.

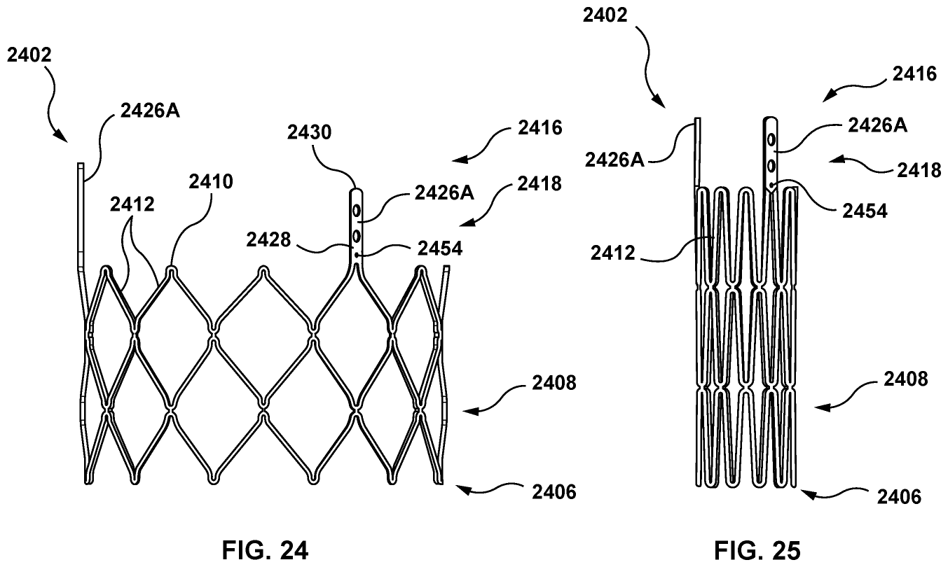

In another embodiment hereof, referring now to FIGS. 24-25, the commissure posts may be formed from a different material than the inflow portion such that each commissure posts 2426A of a stent 2402 is configured to flex radially inward during loading of the transcatheter valve prosthesis. Similar to the stent 102, the stent 2402 has an expanded configuration, which is shown in the side view of FIG. 24, and a non-expanded or crimped configuration, which is shown in the side view of FIG. 25. Although only the stent 2402 is shown, the stent 2402 is configured to be utilized with a prosthetic valve component to form a transcatheter valve prosthesis similar to the transcatheter valve prosthesis 100 described above. The stent 2402 has an inflow portion 2408 and an outflow portion 2418. The stent 2402 is a tubular component defining a central lumen or passageway (not shown in the side views of FIGS. 24-25), and further defines the inflow or proximal end 2406 and the outflow or distal end 2416 of the transcatheter valve prosthesis. The inflow portion 2408 forms the generally tubular shape of the stent 2402 and the outflow portion 2418 includes three commissure bars 2426A longitudinally extending from the inflow portion 2408, as will be described in more detail herein. The inflow portion 2408 of the stent 2402 has the same structure or configuration as the inflow portion 108 of the stent 102 described above, and therefore details thereof are not repeated. When expanded, a diameter of the inflow end 2406 of the stent 2402 is the same as a diameter of the outflow end 2416 of the tubular stent 2402. In an embodiment, the diameter of the inflow and outflow ends may range between 18 and 30 mm in order to accommodate dimensions of the native valve anatomy. The inflow portion 2408 of the stent 2402 may be formed by a laser-cut manufacturing method and/or another conventional stent forming method as would be understood by one of ordinary skill in the art. The cross-section of the stent 2402 may be circular, ellipsoidal, rectangular, hexagonal, square, or other polygonal shape, although at present it is believed that circular or ellipsoidal may be preferable with the transcatheter valve prosthesis being provided for replacement of an aortic valve.

The outflow portion 2418 includes three commissure posts 2426A that longitudinally or axially extend from the inflow portion 2408 and are substantially parallel to the central longitudinal axis of the stent 2402. Similar to commissure posts 126A, each commissure post 2426A is a relatively stiff, axial segment or planar bar having a first end 2428 connected to a crown 2410 of the inflow portion 2408 and an unattached or free second end 2430. The three commissure posts 2426A are circumferentially spaced apart and aligned with and attached to a respective commissure of the three leaflets of the prosthetic valve. A prosthetic valve (not shown in FIGS. 24-25), similar to the prosthetic valve 132 described above, is disposed within and secured to at least the outflow portion 2418 of the stent 2402 at the commissure posts 2426A. The three commissure posts 2426A aid in valve alignment and coaptation. More particularly, the three commissure posts 2426A reinforce or strengthen the commissure region of the prosthetic valve by shaping the leaflets thereof and supporting the leaflets during opening and closing thereof, and thus provide more reliable leaflet coaptation. The shape or configuration of the commissure posts 2426A is not limited to the axial segment or planar bar shown in FIGS. 24-25. For example, in another embodiment hereof (not shown), the commissure posts 2426A may have an upside-down Y shape with two legs longitudinally or axially extending from the inflow portion 2408, with each leg of the upside-down Y shape being connected to a crown 2410 of the inflow portion 2408 and a base of the upside-down Y shape forming an unattached or free second end of the commissure post.

In the embodiment depicted in FIGS. 24-25, the three commissure posts 2426A are the only structures formed at the outflow end 2418 of the stent 2402. The configuration of the stent 2402 maximizes access to the coronary arteries because the commissure posts 2426A are the only structures in the vicinity of the coronary arteries at the outflow portion 2418 of the tubular stent 2402. It is very improbable that the right coronary artery and/or the left main coronary artery will be blocked or jailed by the commissure posts 2426A, and thus there will be clear access to the coronary arteries via a coronary guide catheter once the transcatheter valve prosthesis is deployed in situ. In addition, with the elimination of any outflow crowns at the outflow portion 2418 of the stent 2402, the overall height of the stent 2402 is reduced relative to tubular stent having outflow crowns formed distal to the commissure posts 2426A. A shorter overall height minimizes interaction with aortic anatomy, thereby resulting in less vessel trauma or valve deformation. In another embodiment hereof (not shown), in addition to the three commissure posts 2426A, the outflow portion 2418 of the stent 2402 may also include axial struts that are disposed between adjacent commissure posts 2426A similar to axial struts 126B described in the embodiment of FIGS. 9-10.

As previously stated, the commissure posts 2426A may be formed from a different material than the inflow portion 2408 such that each commissure posts 2426A is configured to flex radially inward during loading of the transcatheter valve prosthesis. More particularly, the inflow portion 2408 of the stent 2402 is made from a first or plastically deformable material such that when expanded by a dilatation balloon, the inflow portion 2408 of the tubular stent 2402 maintains its radially expanded configuration. The inflow portion 2408 of the stent 2402 may be formed from stainless steel or other suitable metal, such as platinum iridium, cobalt chromium alloys such as MP35N, or various types of polymers or other materials known to those skilled in the art, including said materials coated with various surface deposits to improve clinical functionality. The inflow portion 2408 of the stent 2402 is configured to be rigid such that it does not deflect or move when subjected to in-vivo forces, or such that deflection or movement is minimized when subjected to in-vivo forces. In an embodiment, the radial stiffness (i.e., a measurement of how much the inflow portion 2408 of the stent 2402 deflects when subjected to in-vivo forces) of the inflow portion 2408 of the stent 2402 is between 80 N/m and 120 N/m, and the radial stiffness of the inflow portion 2408 of the stent 2402 scaled across the deployed height thereof is approximately 5 N/mm². In an embodiment, the radial stiffness of the inflow portion 2408 of the tubular stent 2402 is greater than 100 N/m. Further, in an embodiment, the device recoil (i.e., a measurement of how much the inflow portion 2408 of the tubular stent 2402 relaxes after balloon deployment) is below 15% and the approximate recoil after deployment is between 1 mm and 2 mm. Further, in an embodiment, the device crush or yield (i.e., the radial force at which the inflow portion 2408 of the stent 2402 yields) is approximately 200 N.

The commissure posts 2426A are made from a second or superelastic material, such as but not limited to Nitinol. The superelastic commissure posts 2426A are configured to flex slightly radially inwardly to reduce stresses observed during valve loading and thereby improve or increase tissue durability of the valve leaflets attached thereto because the strains experienced during valve loading are transferred to the commissure posts 2426A. More particularly, the balloon expandable material of the inflow portion 2408 is stiffer and stronger than the superelastic material of the commissure posts 2426A. The valve leaflets, which are often formed from tissue, are more durable when the portion of the stent to which they are attached is more flexible. As such, the superelastic commissure posts 2426A achieve a balance between stent durability and tissue durability because the inflow portion 2408 maintains its strength and durability while permitting the superelastic commissure posts 2426A to flex inward to increase tissue durability. Stated another way, the superelastic commissure posts 2426A extend the useable life of the balloon expandable transcatheter valve prosthesis.

The superelastic commissure posts 2426A are attached or secured to the distalmost crowns 2410 of the inflow portion 2408 after the inflow portion 2408 is formed by a laser-cut manufacturing method and/or another conventional stent forming method. Each superelastic commissure post 2426A may be attached or secured to a distalmost crown 2410 of the inflow portion 2408 via a rivet 2454 as shown in FIGS. 24-25, or may be attached via another suitable method such as but not limited to welding. The rivets 2454 may be formed from titanium, nickel, or any other suitable metal or alloy.

The commissure posts 2426A have a uniform thickness in the radial direction along a length thereof, or alternatively may be formed with a variable thickness similar to the embodiments of FIGS. 11-15. Further, in another embodiment hereof, the commissure posts 2426A may be relatively longer, wider, and thinner than the struts of the inflow portion such that each commissure posts 2426A of the stent 2402 is configured to flex radially inward during loading of the transcatheter valve prosthesis 100 as described above with respect to FIGS. 19-23.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A transcatheter valve prosthesis comprising:
a stent having a crimped configuration for delivery within a vasculature and an expanded configuration for deployment within a native heart valve, the stent having
an inflow portion formed proximate to an inflow end of the stent, the inflow portion including a plurality of crowns and a plurality of struts with each crown being formed between a pair of opposing struts, a plurality of side openings being defined by the plurality of crowns and the plurality of struts, and
an outflow portion formed proximate to an outflow end of the stent and coupled to the inflow portion, wherein the outflow portion of the stent has exactly three commissure posts, each commissure post longitudinally extending from a crown of the inflow portion and the three commissure posts being circumferentially spaced apart, and wherein a thickness of each commissure post varies along a length thereof such that a first end is relatively thicker than a second end, the first end being coupled to the crown of the inflow portion; and
a prosthetic valve disposed within and secured to at least the outflow portion of the stent, the prosthetic valve being configured to block blood flow in one direction to regulate blood flow through a central lumen of the tubular stent.

2. The transcatheter valve prosthesis of claim 1, wherein the prosthetic valve includes three leaflets and three commissures, each commissure being formed by attached adjacent lateral ends of an adjoining pair of the three leaflets, and wherein the three commissure posts are aligned with and attached to a respective commissure of the three leaflets of the prosthetic valve.

3. The transcatheter valve prosthesis of claim 1 or 2, wherein each commissure post is a planar bar.

4. The transcatheter valve prosthesis of any preceding claim, wherein the thickness of each commissure post is configured to permit each commissure post to flex radially inward during loading of the transcatheter valve prosthesis.

5. The transcatheter valve prosthesis of any preceding claim, wherein each stmt of the inflow portion has a thickness along a length thereof and the thickness of each commissure post at the first end thereof is not greater than the thickness of the strut of the inflow portion.

6. The transcatheter valve prosthesis of any preceding claim, wherein each commissure post has a pre-set curve such that the second end is disposed radially inward relative to the first end.

7. The transcatheter valve prosthesis of claim 6, wherein the second end of each commissure post is disposed between 1 and 2 mm radially inward relative to the first end.

8. The transcatheter valve prosthesis of any preceding claim, wherein each stmt of the inflow portion has a first width along a length thereof and each commissure post has a second width along a length thereof, the first width being less than the second width.

9. The transcatheter valve prosthesis of any preceding claim, wherein the inflow portion is formed from a first material and each commissure post of the outflow portion is formed from a second material, the first material being different than the second material.

10. The transcatheter valve prosthesis of claim 9, wherein the first material is plastically deformable and the second material is superelastic.

11. A transcatheter valve prosthesis comprising:
a stent having a crimped configuration for delivery within a vasculature and an expanded configuration for deployment within a native heart valve, the stent having
an inflow portion formed proximate to an inflow end of the stent, the inflow portion including a plurality of crowns and a plurality of struts with each crown being formed between a pair of opposing struts, a plurality of side openings being defined by the plurality of crowns and the plurality of struts, and
an outflow portion formed proximate to an outflow end of the stent and coupled to the inflow portion, wherein the outflow portion of the stent has exactly three commissure posts, each commissure post longitudinally extending from a crown of the inflow portion and the three commissure posts being circumferentially spaced apart, and wherein each commissure post has a length that is greater than a length of each strut of the inflow portion, each commissure post has a thickness along the length thereof that is less than a thickness of each strut of the inflow portion along the length thereof, and each commissure post has a width that is greater than a width of each strut of the inflow portion; and
a prosthetic valve disposed within and secured to at least the outflow portion of the stent, the prosthetic valve being configured to block blood flow in one direction to regulate blood flow through a central lumen of the stent.

12. The transcatheter valve prosthesis of claim 11, wherein the prosthetic valve includes three leaflets and three commissures, each commissure being formed by attached adjacent lateral ends of an adjoining pair of the three leaflets, and wherein the three commissure posts are aligned with and attached to a respective commissure of the three leaflets of the prosthetic valve.

13. The transcatheter valve prosthesis of claim 11 or 12, wherein each commissure post is a planar bar.

14. The transcatheter valve prosthesis of any of claims 11 to 13, wherein each commissure post has a strength that is greater than a strength of each strut of the inflow portion.

15. The transcatheter valve prosthesis of claim 14, wherein the inflow portion is formed from a first material and each commissure post of the outflow portion is formed from a second material, the first material being different than the second material.

16. The transcatheter valve prosthesis of claim 15, wherein the first material is plastically deformable and the second material is superelastic.

17. A transcatheter valve prosthesis comprising:
a stent having a crimped configuration for delivery within a vasculature and an expanded configuration for deployment within a native heart valve, the stent having
an inflow portion formed proximate to an inflow end of the stent, the inflow portion including a plurality of crowns and a plurality of struts with each crown being formed between a pair of opposing struts, a plurality of side openings being defined by the plurality of crowns and the plurality of struts, wherein the inflow portion is formed from a first material, and
an outflow portion formed proximate to an outflow end of the stent and coupled to the inflow portion, wherein the outflow portion of the tubular stent has exactly three commissure posts, each commissure post longitudinally extending from a crown of the inflow portion and the three commissure posts being circumferentially spaced apart, and wherein each commissure post is formed from a second material different than the first material; and
a prosthetic valve disposed within and secured to at least the outflow portion of the stent, the prosthetic valve being configured to block blood flow in one direction to regulate blood flow through a central lumen of the tubular stent.

18. The transcatheter valve prosthesis of claim 17, wherein the prosthetic valve includes three leaflets and three commissures, each commissure being formed by attached adjacent lateral ends of an adjoining pair of the three leaflets, and wherein the three commissure posts are aligned with and attached to a respective commissure of the three leaflets of the prosthetic valve.

19. The transcatheter valve prosthesis of claim 17 or 18, wherein each commissure post is a planar bar and wherein each commissure post has a length that is greater than a length of each strut of the inflow portion, each commissure post has a thickness along the length thereof that is less than a thickness of each strut of the inflow portion along the length thereof, and each commissure post has a width that is greater than a width of each strut of the inflow portion.

20. The transcatheter valve prosthesis of any of claims 17 to 19, wherein the first material is plastically deformable and the second material is superelastic.
